(19) United States
(12) Patent Application Publication
(10) Pub. No.: US 2008/0014187 A1
(43) Pub. Date: Jan. 17, 2008

(54) Compositions and Methods for Treating Hypertension and Inflammation

(75) Inventor: Bryant Richard Villeponteau, San Diego, CA (US)

Correspondence Address: BRYANT R. VILLEPONTEAU, 4953 SMITH CANYON CT., SAN DIEGO, CA, 92130, US

(73) Assignee: Bryant Richard Villeponteau, San Diego, CA (US)

(21) Appl. No.: 11/457,796

(22) Filed: Jul. 15, 2006

(51) Int. Cl. A61K 38/43; A61K 31/554; A61K 31/401; A61K 31/455; A61K 31/549

(52) U.S. Cl. 424/94.1; 514/690; 514/423; 514/355; 514/211.07; 514/223.2; 514/394

(57) ABSTRACT

The present invention relates to pharmaceutical compositions for reducing essential hypertension and systemic inflammation. While many drugs have been found to treat hypertension, the currently available drugs often do not maintain reduced blood pressure at the preferred norm of 115/75 mm Hg or less throughout a 24 hour period. The current invention provides compositions comprising at least one hypertensive drug combined with the natural product Coenzyme Q10 (ubiquinone or CoQ10), which synergizes with the antihypertensive drugs to maintain low blood pressure throughout the day and night while generating other positive effects on the risks of cardiovascular disease, renal failure, and stroke. CoQ10 also counteracts some of the side effects of some hypertensive drugs such as tiredness, weakness, and/or liver toxicity. The invention further describes therapeutically effective methods for reducing systemic inflammation in hypertensive mammals comprising treatment with an antihypertensive composition that includes at least one angiotensin-converting enzyme inhibitor or angiotensin receptor blocker and CoQ10 (ubiquinone). The invention metrics for reducing systemic inflammation comprise the reduction of serum levels of high sensitivity C-reactive protein (CRP), Interleukin 6 (IL-6), and/or tumor necrosis factor-alpha (TNF-alpha). These antihypertensive-CoQ10 combinations will synergistically reduce both hypertension and systemic inflammation.

### FIELD OF THE INVENTION

The present invention relates to pharmaceutical compositions for reducing essential hypertension and systemic inflammation. Essential or primary hypertension is a medical condition whereby arterial blood pressure is chronically elevated. Essential hypertension is a major cause of aneurysms, arthrosclerosis, heart disease, renal failure, retinopathy, and strokes. While many drugs have been approved to treat hypertension, the currently available drugs often do not maintain reduced blood pressure at the recommended levels throughout a 24 hour period. Typically, more than one antihypertensive drug is often needed to reduce blood pressure to the desired level. Moreover, current drugs often have unwanted side effects such as dizziness, persistent cough, back pain, fatigue, and/or reduced sex drive. The current invention provides compositions comprising at least one of the existing antihypertensive drugs combined with Coenzyme Q10 (ubiquinone or CoQ10), which synergizes with the antihypertensive drugs to maintain low blood pressure throughout the day and night while reducing or neutralizing said side effects.

In addition to the ability to complement antihypertensive drugs in controlling blood pressure, some combinations of antihypertensive drugs with CoQ10 can reduce chronic low-level systemic inflammation. The present invention provides antihypertensive compositions and methods that reduce multiple causes of systemic inflammation as measured by the reduction of serum levels of high-sensitivity C-reactive protein (CRP), interleukin 6 (IL-6), and/or tumor necrosis factor alpha (TNF-alpha) that are systemic indicators of inflammation. While reasonably effective antihypertensive drugs have been around for decades, the present invention presents novel antihypertensive compositions that lower blood pressure along with the three most critical serum cardiovascular indices of systemic inflammation (CRP, IL-6, and TNF-alpha). While patients with high levels of CRP, IL-6, and TNF-alpha have increased risk for cardiovascular and neurodegenerative disease (with or without high cholesterol or high blood pressure), there are presently no good pharmaceutical options for corrective treatment for hypertensive patients who also suffer from chronic systemic inflammation.

### BACKGROUND OF THE INVENTION AND DESCRIPTION OF THE PRIOR ART

Normal Blood pressure is considered 120/80 mm Hg, but some recent data indicate that 115/75 mm Hg or below is the desired norm. Essential or primary hypertension is defined as a chronic elevation of blood pressure of 140/90 mm Hg or above. Although hypertension seldom produces early symptoms, untreated essential hypertension extending over many years damages the heart and the blood vessels in and between the major organs resulting in increasing risks for aneurysms, arthrosclerosis, heart disease, retinopathy, strokes, and kidney damage.

Recently, a US National Committee has defined prehypertension individuals as those with persistent blood pressure between 120/80 mm Hg and 139/89 mm Hg. The prehypertension designation identifies people with higher risk of developing hypertension, along with a higher risk for cardiovascular disease. According to the American Heart Association, some 65 million American adults have hypertension and an additional 59 million adults have prehypertension. Hypertension and prehypertension together thus affect more than half of all adults in the US. Many of these individuals are not being treated for their essential hypertension and most of those under treatment with antihypertensive drugs do not reduce blood pressure to the desired level of 115/75 mmHg throughout a 24 hour day. Therefore, there is an unmet need for better antihypertensive treatments.

The environmental and genetic risk factors for hypertension have been extensively studied. The controllable risk factors include: inactivity; excessive alcohol intake; high sodium or saturated fat diet; low potassium, magnesium, or calcium diet; excess weight or obesity; smoking or other CNS stimulants; and high stress. With the growth of fast food restaurants, overeating, decline of aerobic exercise, and hectic lifestyles, the environmental risk factors for hypertension have been increasing in the developed world. The genetic and uncontrollable risk factors for hypertension include: age, race, sex, and family history of diabetes or hypertension. Because the hypertension risk rises greatly after 50 years of age, essential hypertension is expected to increase significantly as the baby boomers reach retirement age. The increasing obesity and diabetes found in the US also raises the risks of hypertension.

The mechanisms underlying essential hypertension are not completely understood, as essential hypertension arises from unidentified causes. Cardiac output increases early in the development of hypertension and then drops as total peripheral resistance of the blood vessels rises. There are at least four theories to explain this rise in peripheral resistance: (1) the inadequate reduction of sodium by the kidneys of some individuals leads to excess water retention; (2) an overactive rennin-angiotension system leads to vasoconstriction of blood vessels; (3) an overactive sympathetic nervous system leads to chronic stress; and/or (4) direct effects on the vascular smooth muscle cells lead to vasoconstriction of arteries or distension of the veins.

The treatment of prehypertension or mild prehypertension starts with changes in lifestyle to reduce the controllable causes of hypertension such as lack of exercise, smoking, obesity, and poor diet. For those unable to control their hypertension via lifestyle changes, antihypertensive drugs are typically prescribed. Many types of antihypertensive medications are available: angiotensin-converting inhibitors, angiotensin II receptor blockers, diuretics, beta blockers, alpha-blockers, and calcium channel blockers. Which of these drug classes works best for the individual patient is often determined by trial and error. Moreover, doctors often prescribe the use of several classes of antihypertensive drugs simultaneously, as several drugs from different classes are often better than high doses of a single drug class to reach the desired blood pressure goal.

Angiotensin-converting enzyme (ACE) inhibitors reduce blood pressure by inhibiting the formation of angiotensin II that restricts blood vessels. Angiotensin II receptor blockers (ARBs) reduce blood pressure by blocking angiotensin II receptors. The diuretics or “water pills” remove excess sodium to lower blood pressure. Alpha blockers (also called alpha-adrenergic blocking agents) constitute a variety of drugs which block alpha-adrenergic receptors causing muscle contractions in the small arteriole blood vessels. Beta blockers (also called beta-adrenergic blocking agents) block beta-adrenergic receptors to the effects of adrenaline and nor-adrenaline causing the heart to beat slower and less forcefully. Calcium channel blockers (CCBs) act to relax the blood vessels by inhibiting calcium uptake of heart and vascular smooth muscle cells.

Some anti-hypertensive drugs have benefits beyond their ability to reduce blood pressure. For example, studies indicate that inhibiting apoptosis helps in the therapeutic treatment of heart disease [1]. Both oxidative stress and angiotensin II can induce apoptosis in cardiovascular and kidney cells to cause damage to these critical systems. This apoptosis is suppressed by ACE and ARB inhibitors [2-4], which strongly inhibit angiotensin II activity.

Coenzyme Q10 (CoQ10) is an antioxidant and anti-inflammatory agent that has been demonstrated to reduce systolic and diastolic blood pressure in both rodents and humans [5-8]. In eight human clinical trials, CoQ10 decreased the mean systolic blood pressure by 16 mm Hg and the mean diastolic blood pressure by 10 mm Hg [8], which is comparable to the effects of the standard antihypertensive drugs. Since it has been demonstrated that CoQ10 further lowers blood pressure in patients taking antihypertensive drugs [9] and protects against blood pressure variability [10], the current invention recognizes that CoQ10 acts synergistically with the existing anti-hypertensive drugs.

Besides its effectiveness in reducing hypertension, CoQ10 has other positive effects on cardiovascular disease. Clinical trial data show that CoQ10 treatment improves cardiovascular disease conditions like angina, arrhythmia, cardiomyopathies, and congestive heart failure [11-15]. Because of its many benefits and excellent side effect profile, CoQ10 is widely proscribed in Japan and Germany as a stand-alone medication for cardiovascular problems. Therefore, the current invention recognizes that CoQ10 provides supplemental cardiovascular benefits to patients currently taking antihypertensive drugs.

Like ACE and ARB inhibitors, CoQ10 also acts to reduce heart and kidney cell apoptosis by protecting mitochondria [16-22]. Stabilization of mitochondria also reduces free radical generation by mitochondria, lowers LDL oxidation, and extends the lifespan of vascular endothelial, cardiac, liver, and kidney cells. Stabilizing cell mitochondria and lowering LDL oxidation reduces atherosclerotic plaque load and thereby lowers systemic inflammation in the cardiovascular system. Since angiotensin can induce cell apoptosis in vascular endothelial, cardiac, and kidney cells, CoQ10 as an inhibitor of apoptosis is recognized by the current invention as reducing the damaging side effects of hypertension even when combined with drugs not affecting the angiotensin system.

Many hypertensive drugs also have side effects such as tiredness and weakness. The current invention recognizes that CoQ10 may counteract these side effects, as CoQ10 increases the tolerance for exercise [8]. Some hypertensive drugs also pose a risk of liver toxicity, which is counteracted by CoQ10 [23]. Thus, it appears that CoQ10 tends to dampen some of the side effects of the standard hypertensive drugs.

There are no existing patents that conflict with the current invention of combining anti-hypertensive drugs with CoQ10. U.S. Pat. No. 7,015,252 (Fujii et al.) proposes CoQ10 as a composition for lessening oxidative stress. U.S. Pat. No. 6,814,972 (Cavazza) proposes a composition of CoQ10 and propionyl-L-carnitine for the treatment or prevention of vascular disease. Patent application 20040248992 (Julili) proposes a CoQ10 composition with statins to counteract the CoQ10-lowering effects of statins. Patent application 20050154066 (Fujii et al.) proposes treatment with CoQ10 to slow the aging process. Patent application 20060127384 (Capaccioli et al.) uses CoQ10 for preventing or inhibiting apoptosis in cutaneous or adipose cells and tissues. None of this prior art conflicts with the current invention. This is also true for US patent application 20040258674 (Julili) that combines CoQ10 with quercetin among other nutrients to treat hypertension.

The current invention also specifies antihypertensive compositions and methods for treating systemic chronic inflammation, which is not found in the prior art. Mounting evidence suggest that chronic inflammation is a major underlying cause of degenerative disease. For example, high sensitivity C-reactive protein (CRP) is now considered a standard serum blood test for inflammation and a valuable index of future cardiovascular risk that is independent of cholesterol or blood pressure levels [24]. While CRP tests are recommended by an increasing number of physicians as a guide to the patient's future risks for cardiovascular disease, the CRP index suffers from the major drawback that there is currently no recognized pharmaceutical treatment that can be proscribed to lower high CRP values (typically>3 mg/dL). ACE and ARB inhibitors have been reported to reduce serum CRP levels [25-27]. Since CoQ10 is also reported to have an effect on CRP [28], the current invention proposes that compositions combining CoQ10 with ACE or ARB inhibitors should effectively lower CRP.

The current invention recognizes that serum IL-6 and TNF-alpha are also good indicators of systemic inflammation. The ACE inhibitor quinapril has been reported to lower serum IL-6 and TNF-alpha [29, 30]. Other research suggests that CoQ10 also reduces IL-6 and TNF-alpha [31, 32]. Based on these additional anti-inflammatory effects, the current invention proposes that the combination of an ACE or ARB inhibitor with CoQ10 can reduce all three major indicators of systemic inflammation (CRP, IL-6, and TNF-alpha). This synergistic reduction of inflammation indicates that an ACE or ARB inhibitor can be combined with CoQ10 to neutralize generalized inflammation; so as to reduce all cause mortality and the risks of cardiovascular disease.

Therefore, the current invention proposes a novel treatment for chronic inflammation that lowers the serum levels of CRP, IL-6, and TNF-alpha. Using the current invention, testing for CRP levels may become as common as cholesterol tests are now, which would give an independent warning of significant risks of degenerative diseases. By intervening very early in the degenerative process of cardiovascular, diabetic, or neuronal diseases to reduce high serum levels of CRP, IL-6, and TNF-alpha, the current invention has the potential to reduce all-cause mortality risks significantly lower than is currently possible with the control of hypertension alone.

### SUMMARY OF THE INVENTION

The present invention relates to pharmaceutical compositions for reducing essential hypertension and systemic inflammation. Essential hypertension is a major cause of aneurysms, arthrosclerosis, heart disease, renal failure, retinopathy, and strokes. While many drugs have been approved to treat hypertension, the currently available drugs often do not maintain reduced blood pressure at the recommended levels throughout a 24 hour period. The current invention provides compositions comprising at least one hypertensive drug combined with the natural product Coenzyme Q10 (ubiquinone or CoQ10), which synergizes with the antihypertensive drugs to maintain low blood pressure throughout the day and night while generating other positive effects on the risks of cardiovascular disease, renal failure, and stroke. CoQ10 also counteracts some of the side effects of some hypertensive drugs such as tiredness, weakness, and/or liver toxicity.

The invention further describes therapeutically effective methods for reducing systemic inflammation comprising the treatment of mammals in need of such treatment with an antihypertensive composition that includes at least one ACE or ARB inhibitor and CoQ10 (ubiquinone). The invention metrics for reducing systemic inflammation comprise the reduction of serum levels of high sensitivity C-reactive protein (CRP), Interleukin 6 (IL-6), and/or tumor necrosis factor-alpha (TNF-alpha). As an example of a method to reduce said systemic inflammation, the current invention proposes the treatment of humans with a twice daily pharmaceutical composition that contains 20 mg of the ARB inhibitor telmisartan along with 50 mg of CoQ10. As an example of a second method to reduce said systemic inflammation, the current invention proposes the treatment of humans with a twice daily pharmaceutical composition that contains 10 mg of the ACE inhibitor benazepril along with 50 mg of CoQ10. These antihypertensive-CoQ10 combinations should synergistically reduce hypertension and systemic inflammation as indicated by the reduction of blood pressure and plasma levels of CRP, IL-6, and TNF-alpha.

### DETAILED DESCRIPTION OF THE INVENTION

Essential hypertension is often called the ‘silent killer’ and is a leading cause of mortality and morbidity worldwide. While many drugs have been approved to treat hypertension, the currently available drugs often do not maintain reduced blood pressure at the recommended levels throughout a 24 hour period. Essential hypertension (140/90 mm Hg or above) and prehypertension (120/80 mm Hg to 139/89 mm Hg) together affect more than half of all adults in the US. Many individuals have difficulty controlling their hypertension using lifestyle changes and a single class of hypertensive drug and must take more than one class of antihypertensive drug to maintain normal blood pressure. Moreover, many of the individuals under treatment with antihypertensive drugs do not reduce blood pressure to the desired level of 115/75 mmHg throughout a 24 hour day. Therefore, there is an unmet need for more efficacious antihypertensive treatments.

The present invention describes efficacious antihypertensive pharmaceutical compositions comprising: at least one antihypertensive drug combined with the natural product coenzyme Q10 (ubiquinone or CoQ10). CoQ10 is an antioxidant and anti-inflammatory agent that has been demonstrated to reduce systolic and diastolic blood pressure in both rodents and humans. In eight human clinical trials, CoQ10 decreased the mean systolic blood pressure by 16 mm Hg and the mean diastolic blood pressure by 10 mm Hg, which is comparable to the effects of the standard antihypertensive drugs. Since it has been demonstrated that CoQ10 further lowers blood pressure in patients taking antihypertensive drugs and further protects against blood pressure variability, the current invention recognizes that CoQ10 acts synergistically with the existing anti-hypertensive drugs.

Examples of said antihypertensive drugs include: angiotensin-converting enzyme (ACE) inhibitors such as captopril, enalapril, ramipril, quinapril, lisinopril, benazepril, and fosinopril; angiotensin II receptor blockers (ARBs) such as losartan, candesartan, irbesartan, eprosartan, olmesartan, valsartan, and telmisartan; diuretics such as hydrochlorothiozide, bendroflumethiazide, spironolactone, amiloride, triamterene, furosemide, bumetanide, and ethacrynic acid; alpha blockers such as doxazosin, prazosin, and terazosin; beta blockers such as acebutolol, bisoprolal, carteolol, carvedilo, celiprolol, labetalol, mepindolol, metoprolol, oxprenolol, pindolol, atenolol, celiprolol, nadolol, nebivolol, sotalol, timolol, betaxolol, propranolol, and carvedilol; and calcium channel blockers (CCBs) such as amlodipine, felodipine, nicardipine, nifedipine, nimodipine, nisoldipine, nitrendipine, lacidipine, lercanidipine, verapamil, gallopamil, and diltiazem.

Said CoQ10 can be a synthetic product or a natural product isolated from yeast. The preferred form of the CoQ10 product is the pharmaceutical grade “trans isomer” natural product, which is fermented to produce a version identical to the human body's own CoQ10. This natural trans isomer of CoQ10 has been extensively tested in Japanese clinical trials and is the most reliable source of CoQ10. Synthetic CoQ10 is typically processed using tobacco and contains the “cis isomer”, which is not a natural human isomer of CoQ10.

As a composition of the current invention, the ACE inhibitor ramipril at a daily dose of 1.25 mg, 2.5 mg, 5 mg, or 10 mg is combined with 60 to 240 mg of CoQ10. An example of this composition would be a scored tablet containing 2.5 mg of ramipril and 80 to 160 mg of CoQ10. As a method of reducing blood pressure in patients in need of such treatment, this tablet can be taken once daily, or broken in half, so that each half can be taken twice daily.

As a composition of the current invention, the ACE inhibitor benazepril at a daily dose of 5 mg, 10 mg, 20 mg, or 40 mg is combined with 60 to 240 mg of CoQ10. An example of this composition would be a scored tablet containing 10 mg of benazepril and 80 to 160 mg of CoQ10. As a method of reducing blood pressure in patients in need of such treatment, this tablet can be taken once daily, or broken in half, so that each half can be taken twice daily.

As a composition of the current invention, the ACE inhibitor fosinopril at a daily dose of 10 mg, 20 mg, or 40 mg is combined with 60 to 240 mg of CoQ10. An example of this composition would be a scored tablet containing 10 mg of fosinopril and 80 to 160 mg of CoQ10. As a method of reducing blood pressure in patients in need of such treatment, this tablet can be taken once daily, or broken in half, so that each half can be taken twice daily.

As a composition of the current invention, the ACE inhibitor quinapril at a daily dose of 10 mg, 20 mg, or 40 mg is combined with 60 to 240 mg of CoQ10. An example of this composition would be a scored tablet containing 10 mg of quinapril and 80 to 160 mg of CoQ10. As a method of reducing blood pressure in patients in need of such treatment, this tablet can be taken once daily, or broken in half, so that each half can be taken twice daily.

As a composition of the current invention, the ACE inhibitor enalapril at a daily dose of 2.5 mg, 5 mg, 10 mg, or 20 mg is combined with 60 to 240 mg of CoQ10. An example of this composition would be a scored tablet containing 5 mg of enalapril and 80 to 160 mg of CoQ10. As a method of reducing blood pressure in patients in need of such treatment, this tablet can be taken once daily, or broken in half, so that each half can be taken twice daily.

As a composition of the current invention, the ACE inhibitor lisinopril at a daily dose of 2.5 mg, 5 mg, 10 mg, or 20 mg is combined with 60 to 240 mg of CoQ10. An example of this composition would be a scored tablet containing 5 mg of lisinopril and 80 to 160 mg of CoQ10. As a method of reducing blood pressure in patients in need of such treatment, this tablet can be taken once daily, or broken in half, so that each half can be taken twice daily.

As a composition of the current invention, the ARB losartan at a daily dose of 25 mg, 50 mg, or 100 mg is combined with 60 to 240 mg of CoQ10. An example of this composition would be a scored tablet containing 50 mg of losartan and 80 to 160 mg of CoQ10. As a method of reducing blood pressure in patients in need of such treatment, this tablet can be taken once daily, or broken in half, so that each half can be taken twice daily.

As a composition of the current invention, the ARB candesartan at a daily dose of 4 mg, 8 mg, 16 mg, or 32 mg is combined with 60 to 240 mg of CoQ10. An example of this composition would be a scored tablet containing 8 mg of candesartan and 80 to 160 mg of CoQ10. As a method of reducing blood pressure in patients in need of such treatment, this tablet can be taken once daily, or broken in half, so that each half can be taken twice daily.

As a composition of the current invention, the ARB irbesartan at a daily dose of 75 mg, 150 mg, or 300 mg is combined with 60 to 240 mg of CoQ10. An example of this composition would be a scored tablet containing 150 mg of irbesartan and 80 to 160 mg of CoQ10. As a method of reducing blood pressure in patients in need of such treatment, this tablet can be taken once daily, or broken in half, so that each half can be taken twice daily.

As a composition of the current invention, the ARB valsartan at a daily dose of 80 mg, 160 mg, or 320 mg is combined with 60 to 240 mg of CoQ10. An example of this composition would be a scored tablet containing 160 mg of valsartan and 80 to 160 mg of CoQ10. As a method of reducing blood pressure in patients in need of such treatment, this tablet can be taken once daily, or broken in half, so that each half can be taken twice daily.

As a composition of the current invention, the ARB telmisartan at a daily dose of 20 mg, 40 mg, or 80 mg is combined with 60 to 240 mg of CoQ10. An example of this composition would be a scored tablet containing 40 mg of telmisartan and 80 to 160 mg of CoQ10. As a method of reducing blood pressure in patients in need of such treatment, this tablet can be taken once daily, or broken in half, so that each half can be taken twice daily.

As a composition of the current invention, the diuretic hydrochlorothiazide at a daily dose of 12.5 mg, 25 mg, or 50 mg is combined with 60 to 240 mg of CoQ10. An example of this composition would be a scored tablet containing 12.5 mg of hydrochlorothiazide and 80 to 160 mg of CoQ10. As a method of reducing blood pressure in patients in need of such treatment, this tablet can be taken once daily, or broken in half, so that each half can be taken twice daily.

As a composition of the current invention, the alpha-blocker doxazosin at a daily dose of 1 mg, 2 mg, 4 mg, or 8 mg is combined with 60 to 240 mg of CoQ10. An example of this composition would be a scored tablet containing 2 mg of doxazosin and 80 to 160 mg of CoQ10. As a method of reducing blood pressure in patients in need of such treatment, this tablet can be taken once daily, or broken in half, so that each half can be taken twice daily.

As a composition of the current invention, the alpha-blocker terazosin at a daily dose of 1 mg, 2 mg, 5 mg, or 10 mg is combined with 60 to 240 mg of CoQ10. An example of this composition would be a scored tablet containing 2 mg of terazosin and 80 to 160 mg of CoQ10. As a method of reducing blood pressure in patients in need of such treatment, this tablet can be taken once daily, or broken in half, so that each half can be taken twice daily.

As a composition of the current invention, the beta-blocker metoprolol at a daily dose of 50 mg or 100 mg is combined with 60 to 240 mg of CoQ10. An example of this composition would be a scored tablet containing 50 mg of metoprolol and 80 to 160 mg of CoQ10. As a method of reducing blood pressure in patients in need of such treatment, this tablet can be taken once daily, or broken in half, so that each half can be taken twice daily.

As a composition of the current invention, the beta-blocker timilol at a daily dose of 5 mg, 10 mg, or 20 mg is combined with 60 to 240 mg of CoQ10. An example of this composition would be a scored tablet containing 10 mg of timilol and 80 to 160 mg of CoQ10. As a method of reducing blood pressure in patients in need of such treatment, this tablet can be taken once daily, or broken in half, so that each half can be taken twice daily.

As a composition of the current invention, the beta-blocker nadolol at a daily dose of 20 mg, 40 mg, or 80 mg is combined with 60 to 240 mg of CoQ10. An example of this composition would be a scored tablet containing 20 mg of nadolol and 80 to 160 mg of CoQ10. As a method of reducing blood pressure in patients in need of such treatment, this tablet can be taken once daily, or broken in half, so that each half can be taken twice daily.

As a composition of the current invention, the beta-blocker atenolol at a daily dose of 25 mg, 50 mg, or 100 mg is combined with 60 to 240 mg of CoQ10. An example of this composition would be a scored tablet containing 25 mg of atenolol and 80 to 160 mg of CoQ10. As a method of reducing blood pressure in patients in need of such treatment, this tablet can be taken once daily, or broken in half, so that each half can be taken twice daily.

As a composition of the current invention, the CCB amlodipine at a daily dose of 2.5 mg, 5 mg, or 10 mg is combined with 60 to 240 mg of CoQ10. An example of this composition would be a scored tablet containing 5 mg of amlodipine and 80 to 160 mg of CoQ10. As a method of reducing blood pressure in patients in need of such treatment, this tablet can be taken once daily, or broken in half, so that each half can be taken twice daily.

As a composition of the current invention, the CCB felodipine at a daily dose of 2.5 mg, 5 mg, or 10 mg is combined with 60 to 240 mg of CoQ10. An example of this composition would be a scored tablet containing 5 mg of felodipine and 80 to 160 mg of CoQ10. As a method of reducing blood pressure in patients in need of such treatment, this tablet can be taken once daily, or broken in half, so that each half can be taken twice daily.

Hydrochlorothiazide is often combined with other antihypertensive drugs to reduce blood pressure in patients with moderate or difficult-to-control hypertension. One embodiment of the current invention uses hydrochlorothiazide combined with a non-diuretic antihypertensive drug and CoQ10 to generate triple drug compositions. As one example of a triple composition of the current invention, enalapril-hydrochlorothiazide at a daily dose of 5-12.5 mg or 10-12.5 mg can be combined with 80 to 160 mg of CoQ10. As a second example of a triple composition of the current invention, benazepril-hydrochlorothiazide at a daily dose of 10-12.5 mg or 20-12.5 mg can be combined with 80 to 160 mg of CoQ10. As a third example of a triple composition of the current invention, valsartan-hydrochlorothiazide at a daily dose of 80-12.5 mg or 160-25 mg can be combined with 80 to 160 mg of CoQ10. As a fourth example of a triple composition of the current invention, telmisartan-hydrochlorothiazide at a daily dose of 40-12.5 mg or 80-25 mg can be combined with 80 to 160 mg of CoQ10. As a fifth example of a triple composition of the current invention, candesartan-hydrochlorothiazide at a daily dose of 16-12.5 mg or 32-25 mg can be combined with 80 to 160 mg of CoQ10. As a sixth example of a triple composition of the current invention, metoprolol-hydrochlorothiazide at a daily dose of 50-12.5 mg or 100-25 mg can be combined with 80 to 160 mg of CoQ10. As methods of reducing blood pressure in patients in need of such treatment, these tablets can be taken once daily, or broken in half, so that each half can be taken twice daily.

One of the antihypertensive drug-CoQ10 combinations has been tested in a case study. A patient in his 60s with hypertension had been on 5 mg amlodipine in the morning and 40 mg telmisartan in the evening to control his hypertension, but his blood pressure was sometimes high in the early morning on arising and his hypertension would flare up during the day with mild excitement or salty meals. Moreover, the amlodipine-telmisartan combination generated almost daily bouts of postural hypotension and dizziness in the late afternoon. Because of these drug side effects, the patient subsequently dropped the amlodipine and took only a 40 mg daily dose of telmisartan in the evening before retiring. However, his blood pressure was often high (125/85 to 140/90) in the morning on arising and at midday. On one day with a reading of 140/90 at 8 AM, the patient took 30 mg of CoQ10 at 9 AM and his blood pressure sharply dropped to 103/71 by 12 PM. The patient subsequently began taking 30 mg CoQ10 in the morning and evening along with the evening only dose of 40 mg telmisartan. Within a week of this treatment regiment, the patient routinely had readings below 115/75 on arising and at midday. The patient has continued on this regiment for many months and his blood pressure has been consistently low throughout the day (95/62 to 115/75) with only infrequent bouts of postural hypotension. Several years ago, this same patient had also used a benazepril-hydrochlorothiazide (10-12.5 mg dose) combination pill once a day, but could not maintain a consistently low blood pressure on this combination. Therefore, for this patient, the telmisartan-CoQ10 composition of the current invention was a much preferred treatment than the conventional amlodipine-telmisartan or benazepril-hydrochlorothiazide combinations for consistently lowering blood pressure with little or no side effects.

The invention further describes therapeutically effective methods of reducing systemic inflammation comprising the treatment of mammals in need of such treatment with an ACE or ARB inhibitor plus 60 to 240 mg CoQ10. The invention metrics for reducing systemic inflammation comprise the reduction of serum levels of high sensitivity C-reactive protein (CRP), Interleukin 6 (IL-6), and/or tumor necrosis factor-alpha (TNF-alpha).

As one example of a method to reduce said systemic inflammation, the current invention proposes the treatment of humans with a daily pharmaceutical composition that contains 40 mg telmisartan along with 100 mg CoQ10 that synergistically lessen systemic inflammation as indicated by the reduction of plasma levels of hsCRP, IL-6, and TNF-alpha over a period of 3 to 6 months. These three serum tests are independent markers of future risks of cardiovascular or neurodegenerative disease.

As a second example of a method to reduce said systemic inflammation, the current invention proposes the treatment of humans with a daily pharmaceutical composition that contains 8 mg candesartan along with 100 mg CoQ10 that synergistically lessen systemic inflammation as indicated by the reduction of plasma levels of hsCRP, IL-6, and TNF-alpha over a period of 3 to 6 months.

As a third example of a method to reduce said systemic inflammation, the current invention proposes the treatment of humans with a daily pharmaceutical composition that contains 10 mg benazepril along with 100 mg CoQ10 that synergistically lessen systemic inflammation as indicated by the reduction of plasma levels of hsCRP, IL-6, and TNF-alpha over a period of 3 to 6 months.

The compositions of the current invention are typically administered in admixture with suitable pharmaceutical diluents, excipients, or carriers that vary depending on the format of the unit dosage form and consistent with prior art and conventional pharmaceutical practices. The format of the unit dosage form varies dependent on whether oral tablets, capsules, softgels, or liquid units are used.

For instance, for oral administration in the form of a tablet or capsule, the active antihypertensive drug and CoQ10 can be combined with an oral, non-toxic, pharmaceutically acceptable, inert carrier such as lactose, starch, sucrose, glucose, methyl cellulose, magnesium stearate, dicalcium phosphate, calcium sulfate, mannitol, sorbitol, sodium bicarbonate, and the like. For oral administration in liquid form, the oral drug components can be combined with any oral, non-toxic, pharmaceutically acceptable inert carrier such as ethanol, glycerol, water, and the like. Additionally, when desired or necessary, suitable binders, lubricants, disintegrating supplements, and coloring supplements can also be incorporated into the mixture. Suitable binders include starch, gelatin, natural sugars such as glucose or beta-lactose, corn sweeteners, natural and synthetic gums such as acacia, guar, konjack, or sodium alginate, carboxyl-methylcellulose, hydroxypropyl-methylcellulose, polyethylene glycol, waxes, and the like. Lubricants used in these dosage forms include sodium oleate, sodium stearate, magnesium stearate, micro-crystalline cellulose, sodium benzoate, sodium acetate, sodium chloride, silica, and the like. Disintegrators include, without limitation, starch, methylcellulose, agar, bentonite, xanthan gum, and the like. Other components which can be incorporated into the compositions of the present invention include colorings, calcium carbonate, magnesium oxide, magnesium hydroxide, lecithin, flavorings, preservatives, lubricants, flow-enhancers, filling aids, essences, and other aesthetically pleasing components.

Examples of suitable forms for administration include pills, tablets, capsules, and powders. The pill, tablet, capsule, or powder can be coated with a substance capable of protecting the composition from disintegration in the esophagus but will allow disintegration of the composition in the stomach and mixing with food to pass into the patient's small intestine. The polymer can be administered alone or in combination with a pharmaceutically acceptable carrier, diluent or excipient substance, such as a solid, liquid or semi-solid material. Examples of suitable carriers, diluents and excipients include lactose, dextrose, sucrose, sorbitol, mannitol, starches, gum acacia, alginates, tragacanth, gelatin, calcium silicate, cellulose e.g., magnesium carbonate or a phospholipid with which the polymer can form a micelle.

In the present application, the term “pharmaceutically acceptable salts” shall mean non-toxic salts of the compounds employed in this invention which are generally prepared by reacting the free acid with a suitable organic or inorganic base. Examples of such salts include, but are not limited to benzoate, bicarbonate, sodium, calcium, acetate, laurate, malate, maleate, succinate, tannate, tartrate, benzenesulfonate, bisulfate, bitartrate, borate, bromide, calcium edetate, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrobromide, hydrochloride, hydroxynapthoate, iodide, isothionate, lactate, lactobionate, mandelate, mesylate, methylbromide, methylnitrate, methylsulfate, mucate, napsylate, nitrate, oleate, oxalate, pamoate, palmitate, panthothenate, phosphate/diphosphate, polygalacturonate, potassium, salicylate, stearate, subacetate, teoclate, tosylate, and valerate.

In practicing the methods of the invention, combination therapy of antihypertensive drugs with CoQ10 refers to administration of a first therapeutic effective amount(s) of at least one anti-hypertension drug and a second therapeutically effective amount of CoQ10, so as to reduce blood pressure, CRP, IL-6, and/or TNF-alpha. Administration in combination therapy encompasses co-administration of the first and second amounts of the compositions of the combination therapy in a single simultaneous manner, such as in a single capsule or tablet having a fixed ratio of first and second amounts, or in multiple, separate capsules or tablets for each. In addition, such administration also encompasses use of each compound in a sequential manner.

The dosage regimen of anti-hypertension drug to reduce blood pressure is selected in accordance with a variety of factors. These include the age, weight, sex, diet, genetics, and medical condition of the patient, the severity of the disease, the route of administration, pharmacological consideration such as the activity, efficacy, pharmacokinetics and toxicology profiles of the particular compound employed, whether a particular drug delivery system is utilized, and which drug combination treatment is chosen. Thus, the dosage regimen actually employed may vary widely and thus deviate from the examples of dosage regimens or antihypertensive drugs set forth above.

There are several potential advantages of the combination therapies disclosed herein. First, those patients who can not reduce their blood pressure to the desired level of 115/75 mm Hg or below with current drugs should be able to do so using the current invention with little or no undesirable side effects of drug use. Second, patients who can only reduce their blood pressure to the desired level intermittently using current antihypertensive drugs, should be able to consistently maintain blood pressure at 115/75 or below during a 24 hour period. Third, and most significantly, all patients, regardless of their levels of hypertension, should benefit from reducing systemic inflammation, as indicated by reduced serum levels of CRP, IL-6, and/or TNF-alpha. Fourth, liver, kidney, and cardiovascular toxicities of antihypertensive drugs are also reduced using the current invention. In summary, the combination therapies described herein have the potential to revolutionize the treatment of hypertension by administering combination compositions to patients that reduces several major risk factors known to generate higher risk for cardiovascular, cerebral, and renal damage due to hypertension.

The terms ‘patient’ include any mammal, who takes a composition of the current invention for treatment of hypertension and/or systemic inflammation. Administration of antihypertensive drugs with CoQ10 compositions in the current invention includes both self-administration and administration to the patient by another person. Humans are the preferred ‘patents’ for the compositions and methods described herein. However, the term ‘patient’ also includes other mammals besides humans, such as dogs, cats, and horses.

While the many embodiments of the invention have been disclosed above and include presently preferred embodiments, many other embodiments and variations are possible within the scope of the present invention. Accordingly, the details of the preferred embodiments and examples provided are not to be construed as limiting. It is to be understood that the terms used herein are merely descriptive rather than limiting and that various changes, numerous equivalents may be made without departing from the spirit or scope of the claimed invention.

What is claimed is:

1. Therapeutically effective pharmaceutical compositions for reducing hypertension and generalized inflammation comprising at least one antihypertensive drug combined with Coenzyme Q10 (ubiquinone).

2. The pharmaceutical compositions of claim 1 wherein said antihypertensive drugs include: angiotensin-converting enzyme inhibitors such as captopril, enalapril, ramipril, quinapril, lisinopril, benazepril, and fosinopril; angiotensin II receptor blockers such as losartan, candesartan, irbesartan, eprosartan, olmesartan, valsartan, and telmisartan; diuretics such as hydrochlorothiozide, bendroflumethiazide, spironolactone, amiloride, triamterene, furosemide, bumetanide, and ethacrynic acid; alpha blockers such as doxazosin, prazosin, and terazosin; beta blockers such as acebutolol, bisoprolal, carteolol, carvedilo, celiprolol, labetalol, mepindolol, metoprolol, oxprenolol, pindolol, atenolol, celiprolol, nadolol, nebivolol, sotalol, timolol, betaxolol, propranolol, and carvedilol; and calcium channel blockers such as amlodipine, felodipine, nicardipine, nifedipine, nimodipine, nisoldipine, nitrendipine, lacidipine, lercanidipine, verapamil, gallopamil, and diltiazem; and the pharmaceutically acceptable analogs, salts, esters, lactones and isomeric forms thereof.

3. The pharmaceutical compositions of claim 1 wherein said coenzyme Q10 can be a synthetic or natural product, wherein the preferred form of Coenzyme Q10 is the pharmaceutical grade “trans isomer” natural product isolated from yeast, and the pharmaceutically acceptable salts, esters, and lactones thereof.

4. The pharmaceutical compositions of claim 1 wherein an angiotensin-converting enzyme inhibitor such as enalapril at a daily dose of 2.5 mg, 5 mg, 10 mg, or 20 mg is combined with 60 to 240 mg of Coenzyme Q10, and the pharmaceutically acceptable salts, esters, lactones and isomeric forms thereof.

5. The pharmaceutical compositions of claim 1 wherein an angiotensin-converting enzyme inhibitor such as lisinopril at a daily dose of 2.5 mg, 5 mg, 10 mg, or 20 mg is combined with 60 to 240 mg of Coenzyme Q10, and the pharmaceutically acceptable salts, esters, lactones and isomeric forms thereof.

6. The pharmaceutical compositions of claim 1 wherein an angiotensin-converting enzyme inhibitor such as benazepril at a daily dose of 5 mg, 10 mg, 20 mg, or 40 mg is combined with 60 to 240 mg of Coenzyme Q10, and the pharmaceutically acceptable salts, esters, lactones and isomeric forms thereof.

7. The pharmaceutical compositions of claim 1 wherein an angiotensin-receptor blocker such as telmisartan at a daily dose of 20 mg, 40 mg, or 80 mg is combined with 60 to 240 mg of Coenzyme Q10, and the pharmaceutically acceptable salts, esters, lactones and isomeric forms thereof.

8. The pharmaceutical compositions of claim 1 wherein an angiotensin-receptor blocker such as candesartan at a daily dose of 4 mg, 8 mg, 16 mg, or 32 mg is combined with 60 to 240 mg of Coenzyme Q10, and the pharmaceutically acceptable salts, esters, lactones and isomeric forms thereof.

9. The pharmaceutical compositions of claim 1 wherein an angiotensin-receptor blocker such as irbesartan at a daily dose of 75 mg, 150 mg, or 300 mg is combined with 60 to 240 mg of Coenzyme Q10, and the pharmaceutically acceptable salts, esters, lactones and isomeric forms thereof.

10. The pharmaceutical compositions of claim 1 wherein an alpha-blocker such as doxazosin at a daily dose of 1 mg, 2 mg, 4 mg, or 8 mg is combined with 60 to 240 mg of Coenzyme Q10, and the pharmaceutically acceptable salts, esters, lactones and isomeric forms thereof.

11. The pharmaceutical compositions of claim 1 wherein a beta-blocker such as metoprolol at a daily dose of 50 mg or 100 mg is combined with 60 to 240 mg of Coenzyme Q10, and the pharmaceutically acceptable salts, esters, lactones and isomeric forms thereof.

12. The pharmaceutical compositions of claim 1 wherein a beta-blocker such as timilol at a daily dose of 5 mg, 10 mg, or 20 mg is combined with 60 to 240 mg of Coenzyme Q10, and the pharmaceutically acceptable salts, esters, lactones and isomeric forms thereof.

13. The pharmaceutical compositions of claim 1 wherein a calcium channel blocker such as amlodipine at a daily dose of 2.5 mg, 5 mg, or 10 mg is combined with 60 to 240 mg of Coenzyme Q10, and the pharmaceutically acceptable salts, esters, lactones and isomeric forms thereof.

14. The pharmaceutical compositions of claim 1 wherein a calcium channel blocker such as felodipine at a daily dose of 2.5 mg, 5 mg, or 10 mg is combined with 60 to 240 mg of Coenzyme Q10, and the pharmaceutically acceptable salts, esters, lactones and isomeric forms thereof.

15. The pharmaceutical compositions of claim 1 wherein a diuretic such as hydrochlorothiazide at a daily dose of 12.5 mg, 25 mg, or 50 mg is combined with 60 to 240 mg of Coenzyme Q10, and the pharmaceutically acceptable salts, esters, lactones and isomeric forms thereof.

16. A pharmaceutical composition of claim 1 wherein a diuretic such as hydrochlorothiazide at a preferred daily dose of 12.5 mg is combined with a therapeutically effective dose of a non-diuretic antihypertensive drug and 60 to 240 mg of Coenzyme Q10, and the pharmaceutically acceptable salts, esters, lactones and isomeric forms thereof.

17. Pharmaceutical compositions of claim 1 for reducing generalized inflammation in hypertensive patients comprising a therapeutically effective dose of an angiotensin-converting enzyme inhibitor or an angiotensin receptor blocker combined with 60 to 240 mg of Coenzyme Q10, along with the pharmaceutically acceptable salts, esters, lactones and isomeric forms thereof.

18. The compositions of claim 1 wherein each composition is formulated in single or multiple capsules, tablets, softgels, or liquid along with binder, emulsifying, fuller, stabilizing, sustained release, and/or carrier supplements such as: polyethylene glycol, magnesium stearate, magnesium oxide, magnesium hydroxide, calcium carbonate, lecithin, silicone dioxide, starch, stearic acid, microcrystalline cellulose, gelatin, hydroxypropyl-methylcellulose, etc.

19. Methods for treating hypertension and/or systemic inflammation comprising the administration of therapeutically effective doses of any of the compositions in claim 1 to mammals in need of such treatments.

20. The methods of claim 19 wherein the plasma markers of systemic inflammation comprise the serum levels of high-sensitivity C-reactive protein, interleukin 6, and tumor necrosis factor alpha.
